(19) United States
(12) Patent Application Publication
(10) Pub. No.: US 2024/0310382 A1
(43) Pub. Date: Sep. 19, 2024

(54) Standard Characteristic Polypeptide Sequence for Quantitatively Detecting Casein Glycomacropeptide in Polypeptide Product by Mass Spectrometry

(71) Applicants: Wuxi Institute For Food Control, Wuxi (CN); Jiangnan University, Wuxi (CN)

(72) Inventors: Yongwei Feng, Wuxi (CN); Yi Zhang, Wuxi (CN); Chenhui Lu, Wuxi (CN); Haodong Wei, Wuxi (CN); Wenlong Wang, Wuxi (CN); Xiuping Yan, Wuxi (CN); Jing Cui, Wuxi (CN)

(21) Appl. No.: 18/736,894

(22) Filed: Jun. 7, 2024

Related U.S. Application Data
(63) Continuation of application No. PCT/CN2022/128243, filed on Oct. 28, 2022.

(30) Foreign Application Priority Data
Jul. 22, 2022 (CN) 2022108715276

(51) Int. Cl. G01N 33/68

(52) U.S. Cl. CPC G01N 33/6851; G01N 2333/958

(57) ABSTRACT

The present disclosure discloses a standard characteristic polypeptide sequence for quantitatively detecting casein glycomacropeptide in a polypeptide product by mass spectrometry, and belongs to the technical field of inspection and detection. According to the present disclosure, three polypeptides for quantitatively detecting the casein glycomacropeptide are obtained through screening, which have amino acid sequences shown in SEQ ID NO:1, SEQ ID NO:2 and SEQ ID NO:3 respectively, and are suitable for quantitatively analyzing the content of the casein glycomacropeptide in a sample to be detected. According to the present disclosure, accurate quantitative analysis of the casein glycomacropeptide is realized, and the polypeptide sequence can be used for determining whether the content of the casein glycomacropeptide in the product reaches a standard or not.

### REFERENCE TO SEQUENCE LISTING

The instant application contains a Sequence Listing in XML format as a file named “YGHY-2023-40-SEQ.xml”, created on Apr. 19, 2024, of 12,400 bytes in size, and which is hereby incorporated by reference in its entirety.

### TECHNICAL FIELD

The present disclosure relates to a standard characteristic polypeptide sequence for quantitatively detecting casein glycomacropeptide in a polypeptide product by mass spectrometry, specifically relates to a standard characteristic polypeptide sequence of casein glycomacropeptide that can be used for quantitatively detecting casein glycomacropeptide in a sample in combination with mass spectrometry, and belongs to the technical field of inspection and detection.

### BACKGROUND

Casein glycomacropeptide is a glycosylated polypeptide obtained by enzymolysis of κ-casein, which does not contain phenylalanine, contains rich sialic acid in glycosyl, and has the effects of resisting bacteria, diminishing inflammation, inhibiting bacteria, realizing detoxification and promoting brain development of infants. Therefore, the casein glycomacropeptide is used as an ideal food ingredient of formula foods with special medical use for persons with phenylketonuria and formula milk powders for infants. Through qualitative identification and quantitative analysis of the casein glycomacropeptide in the products, whether merchants have adulterate behaviors and whether the quality of casein glycomacropeptide foods reaches standards can be determined.

An amino acid sequence of the casein glycomacropeptide consists of 64 amino acids from methionine at position 106 to valine at position 169 of the κ-casein, and the amino acid sequence has 11 variations. In nature, type A casein glycomacropeptide is dominant, in which Thr at positions 121, 131, 133, 136 and 142 are possible glycosylation sites and are all acetylgalactosamine oxyglycosidic bonds. A back end of the glycosyl mainly has two saccharides, including galactose and sialic acid, and one glycosyl has different composition modes from monosaccharide to tetrasaccharide. The casein glycomacropeptide has a theoretical molecular weight of 7-11 kDa. However, polymers can be formed at specific pH values, which have different apparent molecular weights. Thus, it can be seen that the casein glycomacropeptide has a large molecular weight, a complex glycosylation degree and diverse molecular forms. Therefore, mature methods for accurately quantifying the casein glycomacropeptide are in shortage at present.

Currently, methods for detecting the casein glycomacropeptide mainly include indirect detection methods, such as a resorcinol-hydrochloric acid method for detecting the content of sialic acid so as to indirectly reflect the content of the casein glycomacropeptide, and an electrophoresis method for detecting casein glycomacropeptide polymer and the like. These detection methods have large errors, are susceptible to interference, and cannot be used for accurately detecting the casein glycomacropeptide in casein glycomacropeptide protein foods to reflect the true content of the casein glycomacropeptide. According to a Chinese invention patent application No. CN201810316627.6 with an invention title of “Method for Detecting A1/A2 β-casein by Mass Spectrometry”, key analysis conditions of the method are not suitable for analysis and detection of the casein glycomacropeptide in casein hydrolysates and other polypeptide products. According to a Chinese invention patent application No. CN201810487863.4 with an invention title of “Characteristic Peptide and Method for Detecting Content of A2β-casein in Dairy Product”, liquid chromatography-mass spectrometry used in the method needs to design a treatment process for a specific amino acid fragment of the A2β-casein, and meanwhile, an internal standard peptide with a specific sequence needs to be introduced. The method has the disadvantages of a long detection time, a low detection sample quantity, a high detection cost and the like, and key analysis methods in the method are not suitable for analysis and detection of the casein glycomacropeptide in casein hydrolysates and other polypeptide products. According to a Chinese invention patent application No. CN201911419245.7 with an invention title of “Standard Characteristic Polypeptide Group for Detecting A1 and A2 β-casein in Dairy Product by Mass Spectrometry”, a target peptide fragment extracted by the method and key analysis conditions are also not suitable for analysis and detection of the casein glycomacropeptide in casein hydrolysates and other polypeptide products.

According to a document “Quantitative determination of bovine k-casein macropeptide in dairy products by Liquid chromatography/Electrospray coupled to mass spectrometry (LC-ESI/MS) and Liquid chromatography/Electrospray coupled to tamdem mass spectrometry (LC-ESI/MS/MS)”, an RP-HPLC-ESI-MS technology is used for detecting the contents of type A casein glycomacropeptide, type B casein glycomacropeptide and total casein glycomacropeptides, and advantages and disadvantages of three detection modes, including ultraviolet broad spectrometry (UV), structure illumination microscopy (SIM) and multiple reaction monitoring (MRM), in quantifying the content of the total casein glycomacropeptides are analyzed. However, qualitative analysis of the casein glycomacropeptides is not realized by the method, and only one polypeptide fragment (162-169) obtained after hydrolyzing the casein glycomacropeptides is used for detecting the content of the total casein glycomacropeptides. Therefore, adulterate behaviors of casein glycomacropeptide products may not be detected, and quantitative results may also have errors.

In summary, the methods for detecting the casein glycomacropeptide still have the disadvantages of complicated spectrogram information, difficult analysis and low accuracy. Therefore, it is necessary to establish a method for detecting casein glycomacropeptide that has simpler spectrogram information, easiness in analysis and high accuracy and is based on liquid chromatography-mass spectrometry.

### SUMMARY

The present disclosure provides a standard characteristic polypeptide sequence for quantitatively detecting casein glycomacropeptide in a polypeptide product by mass spectrometry and a method for detecting casein glycomacropeptide in a polypeptide product by using the standard characteristic polypeptide sequence. The polypeptide sequence and the method can be used for quantitatively analyzing the casein glycomacropeptide in a polypeptide product and have the advantages of simple and rapid operation, low cost, high throughput and the like.

According to a first inventive principle of the present disclosure, molecular weight characteristics of casein glycomacropeptide are studied at a whole protein level, and then protease is selected to perform further enzymolysis on the casein glycomacropeptide according to simulated enzyme digestion results to obtain several small non-glycosylated peptide fragments.

According to a second principle of the present disclosure, the small non-glycosylated peptide fragments obtained by enzyme digestion are analyzed and evaluated based on mass spectrometry results, three suitable peptide fragments are selected as potential quantitative peptide fragments for the casein glycomacropeptide, and finally, one peptide fragment is screened out as a quantitative peptide fragment for the casein glycomacropeptide.

According to a third principle of the present disclosure, based on the above principles, quantitative detection of the casein glycomacropeptide in casein hydrolysates and other polypeptide products is completed by detecting a quantitative peptide fragment in the casein hydrolysates and other polypeptide products.

Therefore, the first purpose of the present disclosure is to provide a polypeptide. The polypeptide has an amino acid sequence shown in SEQ ID NO:1, SEQ ID NO:2 or SEQ ID NO:3.

The second purpose of the present disclosure is to provide a method for detecting casein glycomacropeptide by using the polypeptide. The method includes detecting the polypeptide by mass spectrometry.

In one embodiment, the method includes detecting the casein glycomacropeptide by an MRM mass spectrum peak corresponding to 671.0/455.1 (5‰) ions of the polypeptide.

In one embodiment, mobile phase conditions of the mass spectrometry are as follows: at an initial stage, a mobile phase A accounts for 100%; at 40-45 min, the mobile phase A accounts for 70%, and a mobile phase B accounts for 30%; at 45-50 min, the mobile phase A accounts for 20%, and the mobile phase B accounts for 80%; at 50-55 min, the mobile phase B accounts for 100%, and at 55 min, the mobile phase A accounts for 100%;
and the mobile phase A is 100% 0.1 formic acid, and the mobile phase B is acetonitrile.

In one embodiment, the mobile phases are set at a flow rate of 0.1-0.5 mL min⁻¹.

In one embodiment, a chromatographic column is BEH C18 2.1×120 mm 1.7 μm at a column temperature of 35-45° C.

In one embodiment, detection conditions of the mass spectrometry include: a positive ion mode, a capillary voltage of 3.5 kV, a cone voltage of 30 V, an ion source temperature of 100° C., a desolvation gas temperature of 400° C., a desolvation gas flow of 700 lit hr⁻¹, a cone gas flow of 50 lit hr⁻¹, a collision energy of 6/20 V, a mass range of 50-2,000 m/z, and a detector voltage of 1,800 V.

In one embodiment, the mobile phase conditions of the mass spectrometry are as follows: at 0-5 min, the mobile phase A accounts for 98%, and the mobile phase B accounts for 2%; at 5-20 min, the mobile phase A accounts for 70%, and the mobile phase B accounts for 30%; at 20-25 min, the mobile phase A accounts for 70%, and the mobile phase B accounts for 30%; at 25-28 min, the mobile phase A accounts for 98%, and the mobile phase B accounts for 2%; at 28-30 min, the mobile phase A accounts for 98%, and the mobile phase B accounts for 2%;
and the mobile phase A is 100% 0.1 formic acid, and the mobile phase B is acetonitrile.

In one embodiment, the mobile phases are set at a flow rate of 0.1-0.5 mL min⁻¹.

In one embodiment, a chromatographic column is Agilent Advance Peptidemapping 120 Å 2.1×150 mm 2.7 μm at a column temperature of 35-45° C.

In one embodiment, the detection conditions of the mass spectrometry include: a positive ion mode, a scanning mode of MRM, a declustering potential of 30-40 V, an inlet voltage of 8-15 V, an ion source voltage of 4,000-5,000 V, an ion source temperature of 550° C., and a collision energy of 20-50 V.

The present disclosure further provides a method for quantitatively detecting casein glycomacropeptide. The method includes the following specific steps:
(1) pretreatment: treating a sample to be detected with a low-polarity organic solvent, collecting an aqueous phase, terminating a reaction after enzymolysis with protease, and performing filtration with a filter membrane to obtain a sample pretreatment solution;
(2) detection of casein glycomacropeptide: detecting the sample pretreatment solution by the method for detecting casein glycomacropeptide by using the polypeptide to obtain an ion flow chromatogram and a mass spectrogram of a polypeptide in the sample to be detected; and
(3) content calculation: introducing a peak area of the ion flow chromatogram of the polypeptide in step (2) into a standard curve for analysis and calculation so as to obtain the content of the casein glycomacropeptide in the sample.

In one embodiment, a method for constructing the standard curve includes: determining casein glycomacropeptide standard solutions with a series of concentrations by the method above to obtain peak area values, and constructing the standard curve based on the peak area values and the concentrations of the corresponding casein glycomacropeptide standard solutions.

In one embodiment, the low-polarity organic solvent is selected from C5-C12 alkanes or cycloalkanes, C1-C8 halogenated alkanes or mixtures thereof.

In one embodiment, the low-polarity organic solvent is n-hexane.

In one embodiment, the protease is protease K.

In one embodiment, a working concentration of the protease is not less than 0.05 mg mL⁻¹.

In one embodiment, the enzymolysis is performed at 55-65° C. for 8 h.

Beneficial effects are as follows:
1. By the method of the present disclosure, quantitative analysis of the casein glycomacropeptide in a product is realized.
2. HPLC-ESI-QTOF-MS and HPLC-ESI-QqQ MS methods are used in the present disclosure, and relative quantification of the casein glycomacropeptide in a product is realized by identifying a mass spectrum peak of a characteristic polypeptide sequence of the casein glycomacropeptide.
3. According to the method provided by the present disclosure, an internal standard is not required to be additionally added, only pretreatment is required for a sample to be detected to perform detection in an operation process, and the method has the advantages of simple and rapid operation, low cost, high throughput and the like. All reagents and consumables involved in the method of the present disclosure are conventional reagents and consumables that are easy to purchase, which are suitable for performing detection in a majority of laboratories and are easy to popularize.

### BRIEF DESCRIPTION OF FIGURES

FIG. 1 shows (a) an extraction ion flow chromatogram, (b) a primary mass spectrogram corresponding to retention time, (c) an ion amplification diagram at 649.3 m/z and (d) an ion amplification diagram at 1297.6 m/z of a target peptide fragment 1.

FIG. 2 shows (a) fragment ions produced by fragmentation in a peptide chain direction and (b) a secondary mass spectrogram of the target peptide fragment 1;

FIG. 3 shows (a) an extraction ion flow chromatogram, (b) a primary mass spectrogram corresponding to retention time and (c) an ion amplification diagram at 675.3 m/z of a target peptide fragment 2;

FIG. 4 shows (a) fragment ions produced by fragmentation in a peptide chain direction and (b) a secondary mass spectrogram of the target peptide fragment 2;

FIG. 5 shows (a) an extraction ion flow chromatogram, (b) a primary mass spectrogram corresponding to retention time and (c) an ion amplification diagram at 671.3 m/z of a target peptide fragment 3;

FIG. 6 shows (a) fragment ions produced by fragmentation in a peptide chain direction and (b) a secondary mass spectrogram of the target peptide fragment 3;

FIG. 7 shows a standard curve diagram of a standard product of the target peptide fragment 3; and

FIG. 8 shows (a) an extraction ion flow chromatogram and (b) a primary mass spectrogram corresponding to retention time of the target peptide fragment 3 in a milk powder enzymolysis sample to be detected in Example 5.

### DETAILED DESCRIPTION

Examples of the present disclosure are described in detail below. The examples described below are illustrative and are intended only to explain the present disclosure, which shall not be understood as limitations of the present disclosure.

An extraction method of a pure product of casein glycomacropeptide in the present disclosure is referred to the document: Pan X, Chen Y, Zhao P, et al. Highly efficient solid-phase labeling of saccharides within boronic acid functionalized mesoporous silica nanoparticles [J]. Angewandte Chemie International Edition, 2015, 54 (21): 6173-6176. According to the method provided in the document, the relative purity of the pure product of the casein glycomacropeptide provided by the present disclosure is 0.957 according to an ultraviolet detection method.

### Example 1. Simulation of Enzymolysis Peptide Fragments

### 1. Simulation of Enzymolysis Peptide Fragments

A program PeptideMass was used for simulating enzyme digestion of a protein and the mass-to-charge ratio corresponding to mass spectrometry. Simulation conditions were as follows: Single isotope molecular weights were obtained without cysteine treatment, and peptide fragments having a mass number of greater than 500 Da were shown.

### 2. Analysis of Results

An application program PeptideMass was used for simulating enzymolysis of casein glycomacropeptide with a common protein endonuclease, and enzymolysis results were analyzed, as shown in Table 1.

TABLE 1
Fragments obtained by enzymolysis of casein glycomacropeptide

| | Fragment | Amino acid | Molecular | |
|---|---|---|---|---|
| Enzyme | position | sequence | weight | |
| Protease K | 1-9 | QEQNQEQPI | 1112.5 | SEQ ID NO: 4 |
| | 10-17 | RCEKDERF | 1081.5 | SEQ ID NO: 5 |
| | 44-48 | QQKPV | 598.3 | SEQ ID NO: 6 |
| | 52-55 | NNQF | 521.2 | SEQ ID NO: 7 |
| | 86-90 | KSCQA | 535.2 | SEQ ID NO: 8 |
| | 91-96 | QPTTMA | 647.3 | SEQ ID NO: 9 |
| | 97-103 | RHPHPHL | 892.5 | SEQ ID NO: 10 |
| | 109-119 | PPKKNQDKTEI | 1296.7 | SEQ ID NO: 1 |
| | 127-138 | SGEPTSTPTTEA | 1176.5 | SEQ ID NO: 11 |
| | 147-152 | EDSPEV | 674.3 | SEQ ID NO: 2 |
| | 154-159 | ESPPEI | 670.3 | SEQ ID NO: 3 |

As can be seen from Table 1, enzymolysis of the casein glycomacropeptide can be effectively realized by protease K to obtain small peptide fragments. Due to low price, mild enzymolysis conditions and easiness in termination of a reaction and separation, the protease K is an ideal enzyme for enzymolysis of the casein glycomacropeptide. The protease K is used for enzymolysis in the following examples.

### Example 2. Enzymolysis of Casein Glycomacropeptide With Protease K and Screening of Target Peptide Fragments

### 1. Enzymolysis of Casein Glycomacropeptide With Protease K

(a) Preparation of a 20 mg mL⁻¹ protease K stock solution: 20 mg of protease K was weighed, dissolved in 1 mL of pure water, and shaken gently until being completely dissolved, and 50 μL of a resulting solution was packaged in a tube and stored at −20° C.
(b) Preparation of a buffer solution containing 50 mM Tris-HCl (pH=7.5) and 10 mM CaCl₂: 6.06 g of Tris and 1.11 g of CaCl₂ were weighed and dissolved in 900 ml of pure water, concentrated HCl was added dropwise and stirred continuously to adjust the pH to 7.5, and water was added to reach a constant volume of 1,000 mL.
(c) 10 mg of a freeze-dried pure product of casein glycomacropeptide was taken and dissolved in 10 ml of the buffer solution prepared in step (b), and 25 μL of the 20 mg mL⁻¹ protease K stock solution was added for enzymolysis at 58° C. for 8 h.
(d) After the enzymolysis was completed, enzyme deactivation was performed at 95° C. for 10 min, followed by centrifugation at 8,000 rpm for 10 min.
(e) Dialysis was performed with a 300 Da dialysis bag for 2 d, and an internal dialysate was kept and stored at 4° C. to be tested.

### 2. Screening of Target Peptide Fragments

According to sites of the enzymolysis of the casein glycomacropeptide with the protease K, peptide fragments containing five or more amino acid residues were selected. 4 peptide fragments above pentapeptide were produced by the enzymolysis, as shown in Table 2, which were undecapeptide PPKKNQDKTEI at positions 109-119, dodecapeptide SGEPTSTPTTEA at positions 127-138, hexapeptide EDSPEV at positions 147-152 and hexapeptide ESPPEI at positions 154-159, respectively. Due to a glycosylation site contained, the dodecapeptide at positions 127-138 is not an ideal quantitative peptide fragment. Therefore, the undecapeptide PPKKNQDKTEI at positions 109-119 (SEQ ID NO:1), the hexapeptide EDSPEV at positions 147-152 (SEQ ID NO:2) and the hexapeptide ESPPEI at positions 154-159 (SEQ ID NO:3) were selected as 3 standard characteristic polypeptides, respectively. According to site characteristics, it can be seen that the three peptide fragments having an amino acid sequence shown in SEQ ID NO:1, SEQ ID NO:2 or SEQ ID NO:3 are located in an N-terminal, the middle and a C-terminal of the casein glycomacropeptide, respectively.

TABLE 2
Peptide fragments obtained by enzymolysis with
protease K (above pentapeptide)

| Peptide chain | Amino acid | |
|---|---|---|
| position | sequence | Mass number |
| 109-119 | PPKKNQDKTEI | 1296.7 |
| 127-138 | SGEPTSTPTTEA | 1176.5 |
| 147-152 | EDSPEV | 674.3 |
| 154-159 | ESPPEI | 670.3 |

### Example 3. Detection and Analysis of Target Peptide Fragments

### 1. Detection of Enzymolysis Fragments by HPLC-ESI-Q-TOF MS

A mass spectrometer used in this example was: QTRAP 4500 liquid chromatography-mass spectrometry instrument (Ab Sciex of the United States of America).

Liquid chromatography conditions and a mass spectrometry mode used in this example were as follows.

Liquid chromatography conditions were as follows: a chromatographic column was BEH C18 2.1×120 mm 1.7 μm; a mobile phase A was 100% 0.1 formic acid, and a mobile phase B was acetonitrile; gradient elution was as follows: 100% A at an initial stage, 70% A+30% B at 40 min, 20% A+80% B at 45 min, 100% B at 50 min, and 100% A at 55 min; the flow rate was 0.3 ml min⁻¹; the column temperature was 45° C.; and the injection volume was 5 μL.

Mass spectrometry conditions included: a positive ion mode, a capillary voltage of 3.5 kV, a cone voltage of 30 V, an ion source temperature of 100° C., a desolvation gas temperature of 400° C., a desolvation gas flow of 700 lit hr⁻¹, a cone gas flow of 50 lit hr⁻¹, a collision energy of 6/20 V, a mass range of 50-2,000 m/z, and a detector voltage of 1,800 V.

### 2. Retrieval of Polypeptide Sequences

Peptide fragments were retrieved by using a BLAST function of a protein database Uniprot, UniprotKB reference proteomes plus Swiss-Prot was selected as a database, E-threshold was selected as 1,000, Matrix was selected as Auto, Filtering was selected as None, Gapped was selected as yes, and Hits was selected as 1,000. Retrieval results are shown in Table 3, Table 4 and Table 5.

TABLE 3
BLAST retrieval results of a target peptide fragment 1

| | | Amino acid | Matching |
|---|---|---|---|
| Protein source | Position | sequence | degree |
| Original sequence | 109-119 | PPKKNQDKTEI | |
| κ-casein (buffalo) | 109-119 | PPKKNQDKTEI | 100% |
| κ-casein (wild ox) | 76-86 | PPKKNQDKTEI | 100% |
| κ-casein (saigas talarica) | 109-119 | PPKKDQDKTEI | 90.9% |
| κ-casein (sheep) | 109-119 | PPKKDQDKTEI | 90.9% |
| κ-casein (Rupicapra) | 109-119 | PPKKDQDKTEI | 90.9% |
| κ-casein (mountain goat) | 109-119 | PPKKDQDKTEI | 90.9% |
| κ-casein (naemorhedus goral) | 109-119 | PPKKDQDKTEI | 90.9% |
| κ-casein (Capricornis swinhoei) | 109-119 | PPKKDQDKTEI | 90.9% |
| κ-casein (serow) | 109-119 | PPKKDQDKTEI | 90.9% |
| κ-casein (Capricornis crispus) | 109-119 | PPKKDQDKTEI | 90.9% |
| κ-casein (sika deer) | 109-119 | PPKKNQDKTDI | 90.9% |
| κ-casein (musk ox) | 62-72 | PPKKDQDKTEI | 90.9% |
| κ-casein (Ovis dalli) | 62-72 | PPKKDQDKTEI | 90.9% |
| κ-casein (western roe deer) | 62-72 | PPKKNQDKTDI | 90.9% |
| κ-casein (red deer) | 62-72 | PPKKNQDKTDI | 90.9% |
| κ-casein (swamp deer) | 62-72 | PPKKNQDKTDI | 90.9% |
| κ-casein (reindeer) | 62-72 | PPKKNQDKTDI | 90.9% |
| κ-casein (white-tailed deer) | 62-72 | PPKKNQDKTDI | 90.9% |
| κ-casein (mule deer) | 62-72 | PPKKNQDKTDI | 90.9% |
| κ-casein (red brocket deer) | 62-72 | PPKKNQDKTDI | 90.9% |
| κ-casein (elk) | 62-72 | PPKKNQDKTDI | 90.9% |
| κ-casein (wapiti) | 62-72 | PPKKNQDKTDI | 90.9% |
| κ-casein (Sus scrofa) | 100-110 | PPKKNQDKTAI | 90.9% |
| κ-casein (capra) | 109-119 | PPKKDQDKTEV | 81.8% |
| κ-casein (giraffe) | 92-100 | PPKKNQDKTDS | 90% |
| κ-casein (chevrotain) | 109-119 | PPKKDQDKTDT | 80% |
| κ-casein (Collared peccary) | 85-95 | PPKKNQDTTAI | 81.8% |
| κ-casein (Muntiacus reevesi) | 62-72 | PPKKSQDKTDH | 80% |

TABLE 4
BLAST retrieval results of a target peptide fragment 2

| | | Amino acid | Matching |
|---|---|---|---|
| Protein source | Position | sequence | degree |
| Original sequence | 147-152 | EDSPEV | |
| Guanylate kinase | 139-144 | EDSPEV | 100% |
| (Polaromonas) | | | |
| Glutathione reductase | 500-505 | EDSPEI (SEQ ID NO: 12) | 83.3% |
| (arabidopsis thaliana) | | | |
| κ-casein (wild ox) | 104-109 | EASPEV (SEQ ID NO: 13) | 83.3% |

TABLE 5
BLAST retrieval results of a target peptide fragment 3

| | | Amino acid | Matching |
|---|---|---|---|
| Protein source | Position | sequence | degree |
| Original sequence | 121-126 | ESPPEI | |
| κ-casein (wild ox) | 121-126 | ESPPEI | 100% |
| Uncharacterized protein with | 74-79 | ESPPEI | 100% |
| a J structure | | | |
| (Schizosaccharomyces pombe) | | | |

### 3. Analysis and Evaluation of Target Peptide Fragments

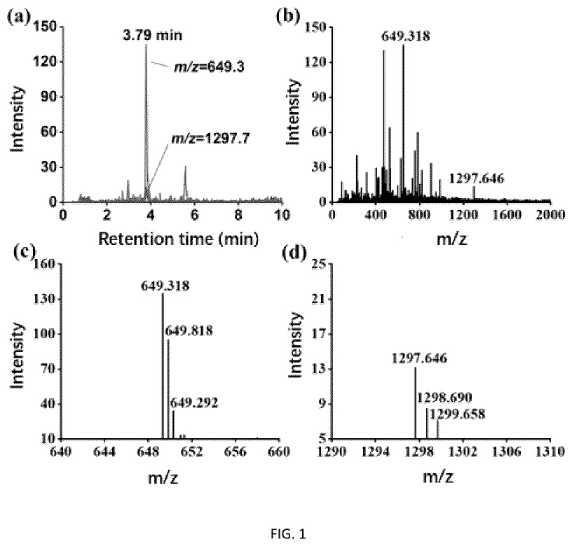

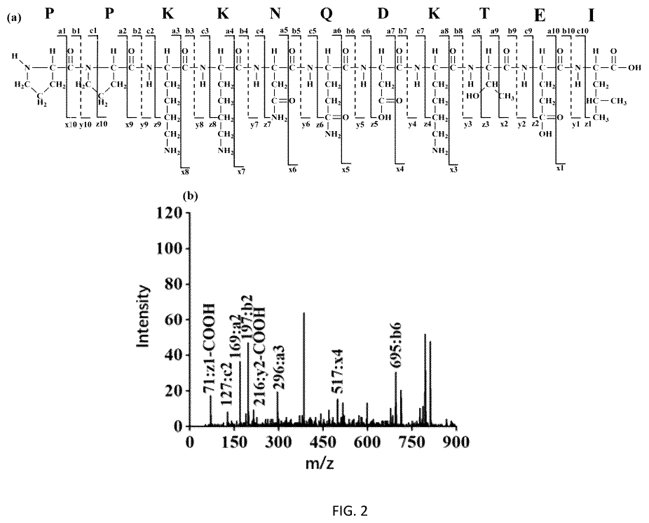

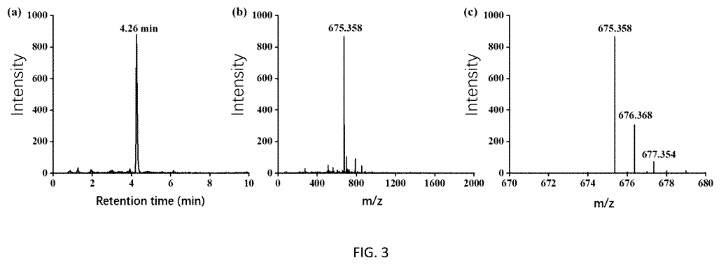

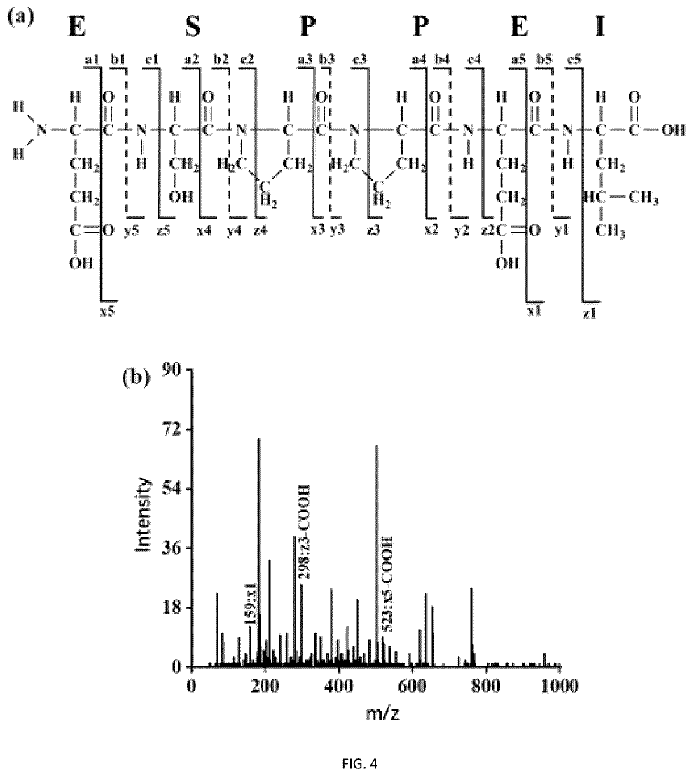

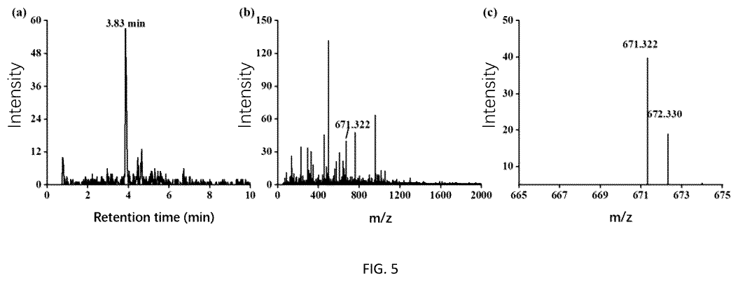

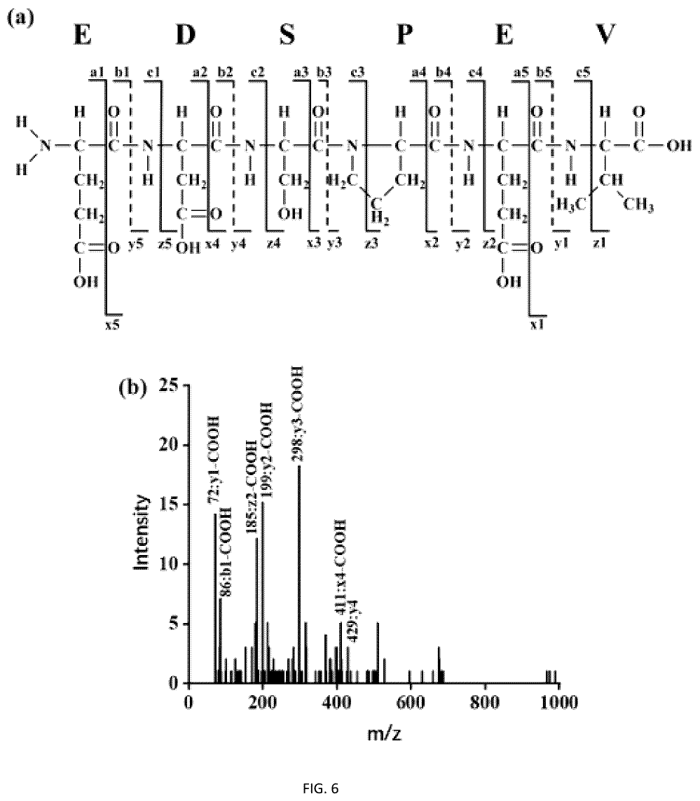

According to mass spectrograms of the target peptide fragment 1, the target peptide fragment 2 and the target peptide fragment 3 as shown in FIG. 1 to FIG. 6, BLAST polypeptide sequence retrieval results as shown in Table 3 to table 5 and simulated enzyme digestion results as shown in Table 1, the 3 target peptide fragments were evaluated in the four terms of ion peak intensity, fragment peak intensity, specificity and hydrolysis degree, respectively. Evaluation results are shown in Table 6.

TABLE 6
Comparison of three peptide fragments

| | | Peak intensity | Peak intensity in | | |
|---|---|---|---|---|---|
| Peptide | Amino acid | in primary mass | secondary mass | | Hydrolysis |
| fragment | sequence | spectrometry | spectrometry | Specificity | degree |
| Target | PPKKNQDKTEI | 148 | 46.8 | Low | 88.10% |
| peptide | | | | | |
| fragment 1 | | | | | |
| Target | EDSPEV | 879 | 18.2 | High | 76.81% |
| peptide | | | | | |
| fragment 2 | | | | | |
| Target | ESPPEI | 57 | 24.8 | High | 42.22% |
| peptide | | | | | |
| fragment 3 | | | | | |

Main characteristic peaks of the 3 target peptide fragments in tandem quadrupole time-of-flight mass spectrometry are shown in Table 7.

TABLE 7
List of main characteristic peaks in tandem
quadrupole time-of-flight mass spectrometry

| | Single charge/ | | Allowable offset |
|---|---|---|---|
| Name | double charge | m/z | range |
| Target peptide fragment 1 | Single charge | 1297.9 | Within ±5‰ |
| Target peptide fragment 1 | Double charge | 649.3 | Within ±5‰ |
| Target peptide fragment 2 | Single charge | 675.3 | Within ±5‰ |
| Target peptide fragment 3 | Single charge | 671.3 | Within ±5‰ |

According to comprehensive evaluation and comparison of the results in Table 3 to Table 7, the target peptide fragment 3 has high specificity. Therefore, the target peptide fragment 3 is used as an ideal peptide fragment for quantitatively detecting the casein glycomacropeptide.

### Example 4. Qualitative and Quantitative Detection of Casein Glycomacropeptide by HPLC-ESI-QqQ MS

### 1. Detection of Casein Glycomacropeptide by HPLC-ESI-QqQ MS

A mass spectrometer used in the present disclosure was: MALDI SYNAPT MS ultra-high performance liquid chromatography tandem quadrupole time-of-flight mass spectrometry instrument (Waters of the United States of America).

Liquid chromatography conditions and a mass spectrometry mode used in this example were as follows.

Liquid chromatography conditions were as follows: a chromatographic column was Agilent Advance Peptidemapping 120 Å 2.1×150 mm 2.7 μm; a mobile phase A was 100% formic acid, and a mobile phase B was acetonitrile; gradient elution was as follows: 98% A+2% B at 0-5 min, 70% A+30% B at 5-20 min, 70% A+30% B at 20-25 min, 98% A+2% B at 25-28 min, and 98% A+2% B at 28-30 min; the flow rate was 0.3 mL min⁻¹; the column temperature was 40° C.; and the injection volume was 10 μL.

A mass spectrometry mode included: a positive ion mode, a scanning mode of MRM, a declustering potential of 35 V, an inlet voltage of 10 V, an ion source voltage of 4,500 V, an ion source temperature of 550° C., a collision energy of 40 V, an ion source gas 1 of 60 psi, and an ion source gas 2 of 40 psi.
(1) Preparation of a standard product of the target peptide fragment 3:
\textcircled{1} Synthesis sequence: from a C-terminal to an N-terminal of a sequence, steps were as follows:
a. A n equivalent amount of resin was weighed and placed into a reactor, dichloromethane (DCM) was added for swelling for half an hour, and then the DCM was removed; a 2 n equivalent amount of a first amino acid in the sequence of the target peptide fragment 3 was added, and a 2 n equivalent amount of diisopropylethylamine (DIEA) and appropriate amounts of dimethylformamide (DMF) and DCM (appropriate amounts mean that the resin can be fully bubbled) were added to enable a nitrogen bubbling reaction of the DIEA, the DMF and the DCM for 60 min; and then about a 5n equivalent amount of methanol was added for a reaction for half an hour, and a reaction solution was removed and washed with DMF and methanol (MEOH).
b. A 2 n equivalent amount of a second amino acid in the sequence of the target peptide fragment 3, a 2 n equivalent amount of 1-hydroxybenzotriazole tetramethylhexafluorophosphate (HBTU) and DIEA were added into a reactor to carry out a nitrogen bubbling reaction for 30 min, a liquid was washed, detection was performed with ninhydrin, and then terminals were sealed with pyridine and acetic anhydride; and finally, washing was performed, an appropriate amount of a cap removal solution was added to remove a 9-fluorenylmethyloxycarbonyl (Fmoc) protective group, washing was performed, and detection was performed with ninhydrin.
c. Other amino acids in the sequence of the target peptide fragment 3 were sequentially added by the way in step b, and various modifications were performed.
d. The resin was removed from a reaction column after being dried with nitrogen, and then poured into a flask; and then, a cutting solution (consisting of 95% trifluoroacetic acid (TFA), 2% ethanedithiol, 2% triisopropylsilane and 1% water) that was about 10 ml/g of the resin was added into the flask and shaken to filter out the resin.
e. A filtrate was obtained, a large amount of ethyl ether was added into the filtrate to precipitate a crude product, and then centrifugation and washing were performed to obtain a crude product of the sequence.
\textcircled{2} Purification of a polypeptide: The crude product was purified by high performance liquid chromatography.
\textcircled{3} Freeze-drying of a polypeptide: A purified liquid was concentrated and freeze-dried in a freeze-drying machine to obtain a white powder, namely, a standard product of the target peptide fragment 3.

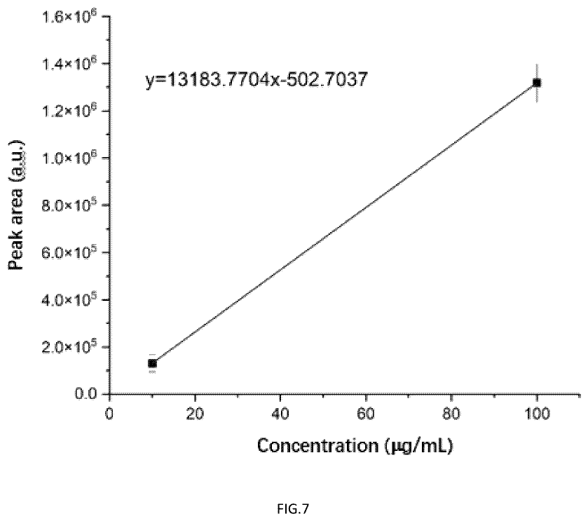

The standard product of the target peptide fragment 3 was prepared by Shanghai Qiangyao Biological Technology Co., Ltd.
(2) Preparation of standard solutions: 5 mg of the standard product of the target peptide fragment 3 prepared in step (1) was dissolved in 1 mL of distilled water and diluted step by step to obtain standard solutions of the target peptide fragment 3 with a concentration of 10 μg mL⁻¹ and 100 μg mL⁻¹ respectively, and a standard curve of the target peptide fragment 3 was drawn according to analysis and detection by liquid chromatography tandem mass spectrometry. The standard curve of the obtained standard product of the target peptide fragment 3 is shown in FIG. 7, where y=13183.7704x-502.7037.
(3) Preparation of a sample: 5-10 g of a food sample was weighed and dissolved in 30 mL of deionized water, 10 mL of n-hexane was added for shaking to remove fat, standing was performed until a solution was layered, an organic phase was removed, extraction was repeated for 3 times, and an aqueous phase finally obtained was precooled and freeze-dried in a freeze-drying machine to be tested.
(4) Preparation of a sample buffer solution: Preparation of a buffer solution containing 50 mmol L⁻¹ Tris-HCl with a pH value of 7.5 and 10 mmol L⁻¹ CaCl₂: 6.06 g of Tris and 1.11 g of CaCl₂ were weighed and dissolved in 900 mL of pure water, concentrated HCl was added dropwise and stirred continuously to adjust the pH to 7.5, and water was added to reach a constant volume of 1,000 mL.
(5) Enzymolysis with protease K: 100 mg of the freeze-dried sample was dissolved in 5 mL of the sample buffer solution, and 25 μL of a 20 mg mL⁻¹ protease K stock solution was added for enzymolysis at 58° C. for 8 h. After the enzymolysis was completed, enzyme deactivation was performed at 95° C. for 10 min, centrifugation was performed at 4,310 g for 10 min, a supernatant was remained, dialysis was performed with a 300 Da dialysis bag for 2 d, and an internal dialysate was remained and stored at 4° C. to be tested.
(6) An enzymolysis solution of the sample to be detected was filtered with a 0.22 μm aqueous phase filter membrane and then detected by HPLC-ESI-QqQ MS to obtain an ion flow chromatogram and a mass spectrogram of a polypeptide of the sample to be detected. Then, quantitative determination was performed, and a specific method was as follows: a peak area of the ion flow chromatogram of the peptide fragment shown in SEQ ID NO:3 of the sample to be detected was introduced into the standard curve obtained in step (2) for analysis and calculation, and calculation was performed by a calculation formula so as to obtain the content of the casein glycomacropeptide in the sample.

### Example 5 Quantitative Test of Casein Glycomacropeptide in a Milk Powder Enzymolysis Sample

With reference to the method described in Example 4, a quantitative test was performed on the casein glycomacropeptide in a milk sample.

The milk sample in this example was obtained from Xinnong Tianshan whole milk powder.

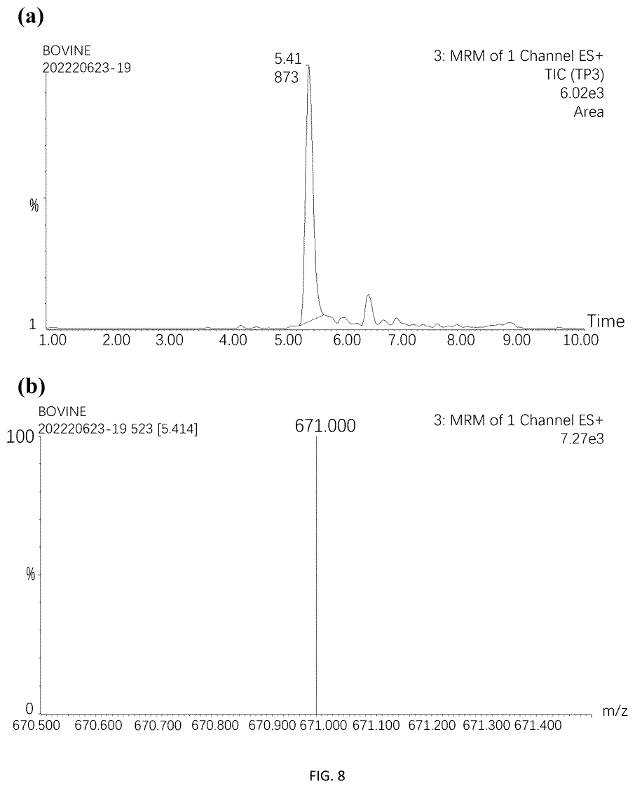

An ion flow chromatogram and a mass spectrogram of a polypeptide of the milk sample to be tested are shown in FIG. 8. A peak area value of the ion flow chromatogram of the target peptide fragment 3 of the sample to be tested (876 as shown in FIG. 8) was introduced into a standard curve shown in FIG. 7 for analysis and calculation, and then calculation was performed by a calculation formula so as to obtain that the content of the casein glycomacropeptide in the sample was 0.1046 μg/mL.

Although the present disclosure has been disclosed as preferred examples above, the examples are not intended to limit the present disclosure, and various changes and modifications can be made by anyone familiar with the art without departing from the spirit and scope of the present disclosure. Therefore, the scope of protection of the present disclosure shall be defined by the claims.

What is claimed is:

1. A method for detecting casein glycomacropeptide, wherein the method comprises detecting a polypeptide having the amino acid sequence set forth in any one of SEQ ID NO:1, SEQ ID NO:2 or SEQ ID NO:3 by mass spectrometry.

2. The method according to claim 1, wherein the method comprises detecting the casein glycomacropeptide by an MRM mass spectrum peak corresponding to 671.0/455.1 (5‰) ions of the polypeptide having the amino acid sequence set forth in any one of SEQ ID NO:1, SEQ ID NO:2 or SEQ ID NO:3.

3. The method according to claim 1, wherein mobile phase conditions of the mass spectrometry are as follows: at an initial stage, a mobile phase A accounts for 100%; at 40-45 min, the mobile phase A accounts for 70%, and a mobile phase B accounts for 30%; at 45-50 min, the mobile phase A accounts for 20%, and the mobile phase B accounts for 80%; at 50-55 min, the mobile phase B accounts for 100%, and at 55 min, the mobile phase A accounts for 100%; alternatively, at 0-5 min, the mobile phase A accounts for 98%, and the mobile phase B accounts for 2%; at 5-20 min, the mobile phase A accounts for 70%, and the mobile phase B accounts for 30%; at 20-25 min, the mobile phase A accounts for 70%, and the mobile phase B accounts for 30%; at 25-28 min, the mobile phase A accounts for 98%, and the mobile phase B accounts for 2%; at 28-30 min, the mobile phase A accounts for 98%, and the mobile phase B accounts for 2%;
and the mobile phase A is 100% 0.1 formic acid, and the mobile phase B is acetonitrile.

4. The method according to claim 3, wherein the mobile phases are set at a flow rate of 0.1-0.5 mL min⁻¹, and detection conditions of the mass spectrometry comprise: a positive ion mode, a scanning mode of MRM, a declustering potential of 30-40 V, an inlet voltage of 8-15 V, an ion source voltage of 4,000-5,000 V, an ion source temperature of 550° C., and a collision energy of 20-50 V.

5. The method according to claim 1, wherein the method comprises the following specific steps:
(1) pretreatment: treating a sample to be detected with a low-polarity organic solvent, collecting an aqueous phase, terminating a reaction after enzymolysis with protease, and performing filtration with a filter membrane to obtain a sample pretreatment solution;
(2) detection of casein glycomacropeptide: detecting the sample pretreatment solution to obtain an ion flow chromatogram and a mass spectrogram of a polypeptide in the sample to be detected; and
(3) content calculation: introducing a peak area of the ion flow chromatogram of the polypeptide in step (2) into a standard curve for analysis and calculation so as to obtain the content of the casein glycomacropeptide in the sample to be detected.

6. The method according to claim 5, wherein a method for constructing the standard curve comprises: determining casein glycomacropeptide standard solutions with a series of concentrations to obtain peak area values, and constructing the standard curve based on the peak area values and the concentrations of the corresponding casein glycomacropeptide standard solutions.

7. The method according to claim 5, wherein in step (1), the low-polarity organic solvent is selected from C5-C12 alkanes or cycloalkanes, C1-C8 halogenated alkanes or mixtures thereof.

8. The method according to claim 7, wherein the low-polarity organic solvent is n-hexane.

9. The method according to claim 5, wherein in step (1), the protease is protease K.

10. The method according to claim 5, wherein a working concentration of the protease is not less than 0.05 mg mL⁻¹, and the enzymolysis is performed at 55-65° C. for 8 hours.


SEQUENCE LISTING (13 sequences)

SEQ ID NO: 1 - protein, 11 aa, synthetic construct
PPKKNQDKTEI

SEQ ID NO: 2 - protein, 6 aa, synthetic construct
EDSPEV

SEQ ID NO: 3 - protein, 6 aa, synthetic construct
ESPPEI

SEQ ID NO: 4 - protein, 9 aa, synthetic construct
QEQNQEQPI

SEQ ID NO: 5 - protein, 8 aa, synthetic construct
RCEKDERF

SEQ ID NO: 6 - protein, 5 aa, synthetic construct
QQKPV

SEQ ID NO: 7 - protein, 4 aa, synthetic construct
NNQF

SEQ ID NO: 8 - protein, 5 aa, synthetic construct
KSCQA

SEQ ID NO: 9 - protein, 6 aa, synthetic construct
QPTTMA

SEQ ID NO: 10 - protein, 7 aa, synthetic construct
RHPHPHL

SEQ ID NO: 11 - protein, 12 aa, synthetic construct
SGEPTSTPTTEA

SEQ ID NO: 12 - protein, 6 aa, synthetic construct
EDSPEI

SEQ ID NO: 13 - protein, 6 aa, synthetic construct
EASPEV